(19) United States
(12) Patent Application Publication
(10) Pub. No.: US 2012/0067970 A1
(43) Pub. Date: Mar. 22, 2012

(54) Air freshener

(75) Inventor: Hami Hossein, Sivizzo (VI) (IT)

(73) Assignee: H.D. TRADING UNIPERSONALE, Vicenza (IT)

(21) Appl. No.: 13/264,631

(22) PCT Filed: Apr. 16, 2010
(86) PCT No.: PCT/IB10/51665

§ 371 Date: Dec. 9, 2011

(30) Foreign Application Priority Data
Apr. 16, 2009 (IT) VI2009U000015

(51) Int. Cl. A61L 9/04

(52) U.S. Cl. 239/34

(57) ABSTRACT

An air freshener (1), particularly for motor vehicles, comprises a support element (2) adapted to be at least partially inserted into an air grille (G) or the like of a space to be deodorized; at least one decorative element (3) is associated with the support element (2) so that it is visible from outside the grille (G) when the support element (2) is in its operative position. The decorative element (3) is made from a first polymer material, which includes a thermoplastic polymer matrix with a scent agent designed to be spread in the environment.

### FIELD OF THE INVENTION

The present invention generally finds application in the field of hygiene products and relates to an air freshener for closed spaces, particularly for interior compartments of motor vehicles, and the like.

### BACKGROUND ART

Various air fresheners are known, particularly for interior compartments of motor vehicles or the like, which consist of a scented portion adapted to be removably secured to an air grille, so that a forced air flow therethrough causes the scent to spread in the environment.

One example of such prior art devices is disclosed in WO03/097114, in which the air freshener has a support element adapted to be at least partially inserted in a concealable fashion into an air grille of a space to be deodorized, and a projecting head associated with the support element in such a position as to be visible from outside the grille with the support element is inserted therein.

The head of this air freshener is composed of a base element, particularly a polyester-based thermoplastic elastomer, impregnated with synthetic scent materials or essential oils, with a decorative element, particularly a transfer film.

This prior art air freshener involves a rather difficult and expensive fabrication process.

Furthermore, the scent tends to fade in a very short time and becomes ineffective, sometimes even before actual use. Also, during use, the decorative element tends to be peeled off.

### DISCLOSURE OF THE INVENTION

The object of the present invention is to provide an air freshener, particularly for interior compartments of motor vehicles, that is highly efficient and relatively cost-effective.

A particular object is to provide an air freshener that affords a particularly convenient and simple use.

Another particular object is to provide an air freshener that maintains its effectiveness for considerably longer times than prior art air fresheners.

Yet another object of the present invention is to provide an air freshener whose scent properties do not tend to decrease before use.

These and other objects, as better explained hereafter, are fulfilled by an air freshener as defined in claim 1.

In its basic form, the air freshener, that is particularly adapted to scent the interior compartment of a motor vehicle, may include a support element, designed to be at least partially inserted in a concealable manner into the air grille of a space to be deodorized, at least one first decorative element being provided, which is preferably removably associated to the support element, so as to be visible from outside the grille when the support element is in its operating position, i.e. at least partially inserted in a concealable manner in the grille.

In a preferred, non exclusive embodiment, the first decorative element may be made from a first polymer material, including a thermoplastic, e.g. EVA-based polymer matrix, with a scent agent dispersed therein, that may be selected from the group comprising natural and synthetic scent materials, natural essences and essential oils, for deodorizing the environment.

With this configuration, a single element may have both air freshening and decorative functions, whereby the air freshener can be fabricated in a more convenient and simpler manner as compared with those of the prior art.

As used herein, the term “decorative element” or derivatives thereof is intended to designate an element that at least partially represents a figure or a fancy picture.

As used herein, the term “matrix” of a polymer or derivatives thereof, shall be intended to indicate a polymer material that can provide the molecular structure of the final product.

As used herein, the term “-based matrix” or derivatives thereof, preceded by the name of a polymer material, shall be intended to indicate a polymer material capable of providing the molecular structure of the polymer material whose name precedes the term “-based matrix” to the final product.

For instance, the first decorative air freshening element may be formed by molding of the first thermoplastic material. Therefore, the first decorative air freshening element may have any shape, preferably an irregular shape, such as the shape of a four-leaved clover, a ladybug, a bee, a flower, a frog, a gecko or the like.

Preferably, the first material that constitutes the first decorative air freshening element only consists of the thermoplastic polymer matrix and the scent agent.

Advantageously, the scent agent may represent from 20% to 40%, preferably from 25% to 35% by weight based on the total weight of the first material, whereas the polymer matrix may represent from 80% to 60%, preferably from 75% to 65% by weight based on the total weight of the first material.

In a preferred, non exclusive embodiment of the invention, the first decorative air freshening element may have a composition including 70%-72% polymer matrix and 28%-30% scent agent, with % still being intended as percent by weight based on the total weight of the first material.

Conveniently, the first decorative air freshening element may have a first pigmentation, e.g. yellow, a second decorative element being provided having a second pigmentation, different from the first, e.g. black, which is associated with the first decorative air freshening element so as to be at least partially visible from outside by a user.

Possibly, the association of the first decorative air freshening element and the second decorative element may be of removable type.

Conveniently, the first decorative air freshening element and the second decorative element may have mating shapes.

This will provide an air freshener with a multi-colored front portion.

In a preferred, non exclusive embodiment of the invention, the support element may include at least one substantially elongate arm, which may in turn include at least one second air freshening element associated therewith to increase the scent power of the air freshener.

Advantageous embodiments of the invention are defined in accordance with the dependent claims.

### BRIEF DESCRIPTION OF THE DRAWINGS

Further features and advantages of the invention will become more apparent upon reading the following detailed description of a few preferred non exclusive embodiments of an air freshener of the present invention, which are described by way of a non limiting example with the help of the accompanying drawings in which:

FIG. 1 is a perspective view of an air freshener of the invention according to a first preferred embodiment;

FIG. 2 is a perspective view of an air freshener of the invention according to a second preferred embodiment;

FIG. 3 is a top view of the support 2;

FIGS. 4 and 5 are perspective exploded views of an air freshener of the invention according to a third preferred embodiment.

### DETAILED DESCRIPTION OF A PREFERRED EMBODIMENT

Referring to the above figures, the air freshener of the invention, generally designated by numeral 1, may be used to deodorize or scent spaces such as interior compartments of vehicles in general or any other space in which an air grille is provided for forced air flow.

FIGS. 2 and 3 show two embodiments of the air freshener 1, which may consist in both cases of a support element 2 and a decorative element 3 removably associated therewith. On the other hand, the support element 2 and the decorative element 3 may be stably associated together, e.g. using an adhesive.

The support element 2 may be at least partially inserted in a concealable manner into an air grille of the space to be deodorized, which is schematically indicated in the figures by broken lines, and designated by the letter G.

The decorative element 3 may be mounted to the support element 2 so as to be visible from outside the grille G when the support element 3 is inserted therein.

The decorative element 3 may be formed by molding of a polymer material consisting of 70%-72% EVA (Ethylene-Vinyl-Acetate)-based thermoplastic polymer matrix and 28%-30% scent agent dispersed in the matrix to deodorize the environment. Thus, the decorative element 3 will also have a deodorizing function.

The support element 2, that may be formed of any plastic, composite or metal material, may include a pair of substantially parallel elongate arms 4, 4′ integral with each other at a transverse end plate 5.

Each arm 4, 4′ may have a corresponding second air freshening element 6, 6′, thereby increasing the deodorizing power of the air freshener 1. Each air freshening element 6, 6′ may be removably associated to its respective arm 4, 4′, e.g. by male-female means of known type.

Each air-freshening element 6, 6′ may, but is not required to have, the above described composition. The air freshening elements 6, 6′ may be different in shape and/or composition.

Each arm 4, 4′ may have corresponding means for attachment to the air grille, which may consist, in the preferred, non exclusive embodiment of the figures, of transverse bulges, generally designated by numerals 7, 7′, longitudinally offset and formed on the inner side of the arms 4, 4′.

The bulges 7, 7′ may be located in such axial positions that the bulges 7 of the arm 4 face the bulges 7′ of the other arm 4′.

The end plate 5 may have any regular or irregular shape, and may be also shaped according to a particular decorative design.

The plate 5 may be either formed of one piece with the arms 4, 4′ or integrally or removably attached thereto.

In a preferred embodiment, the plate 5 may include male-female means for removably associating the decorative air freshening element 3 with the support element 2, e.g. a pair of elongate pins 8, 8′ adapted to be inserted into corresponding female seats 9, 9′ formed on the decorative element 3, possibly at its rear portion.

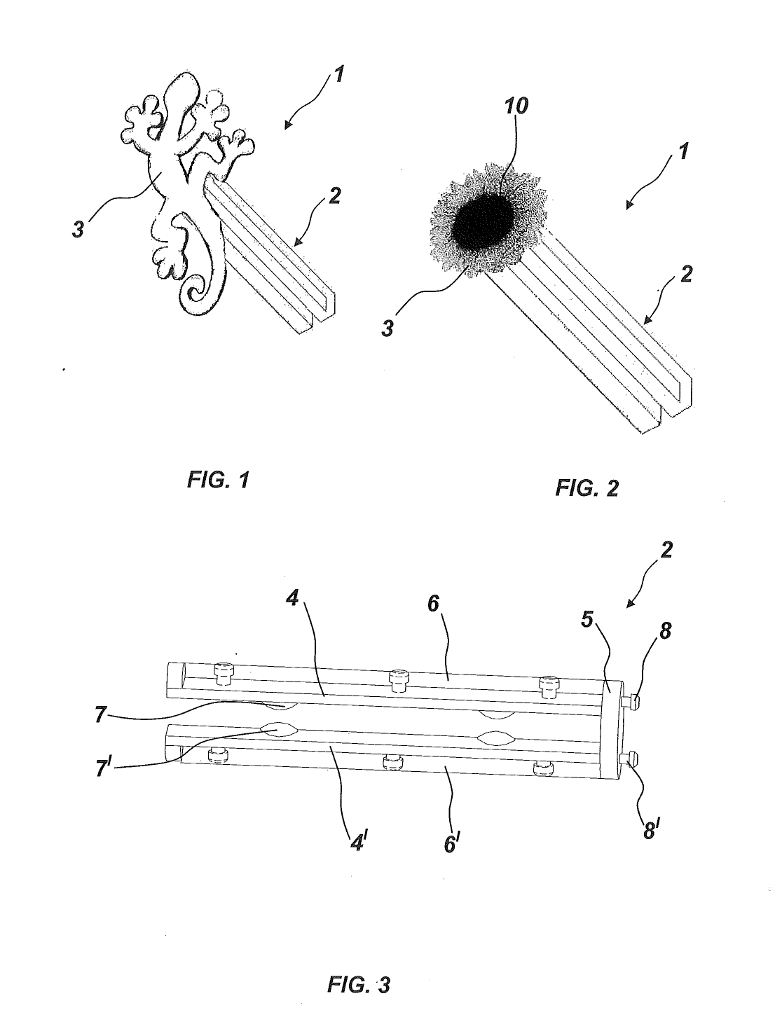

In the embodiment as shown in FIG. 1, the decorative air freshening element 3 may have a single pigmentation, and form the single decorative element of the air freshener 1, here representing a gecko.

On the other hand, in the embodiment as shown in FIG. 2, the air freshener 1 may include a first decorative air freshening element 3 and a second decorative element 10, associated with the first element, possibly in a removable manner, to form the figure of a flower.

The two elements 3 and 10 may have different pigmentations, e.g. a yellow pigmentation for the element 3 and a pink pigmentation for the element 10. Therefore, the second decorative element 10 may be conveniently frontally associated with the first element, to be at least partially visible from outside by a user, thereby providing an overall polychromatic effect.

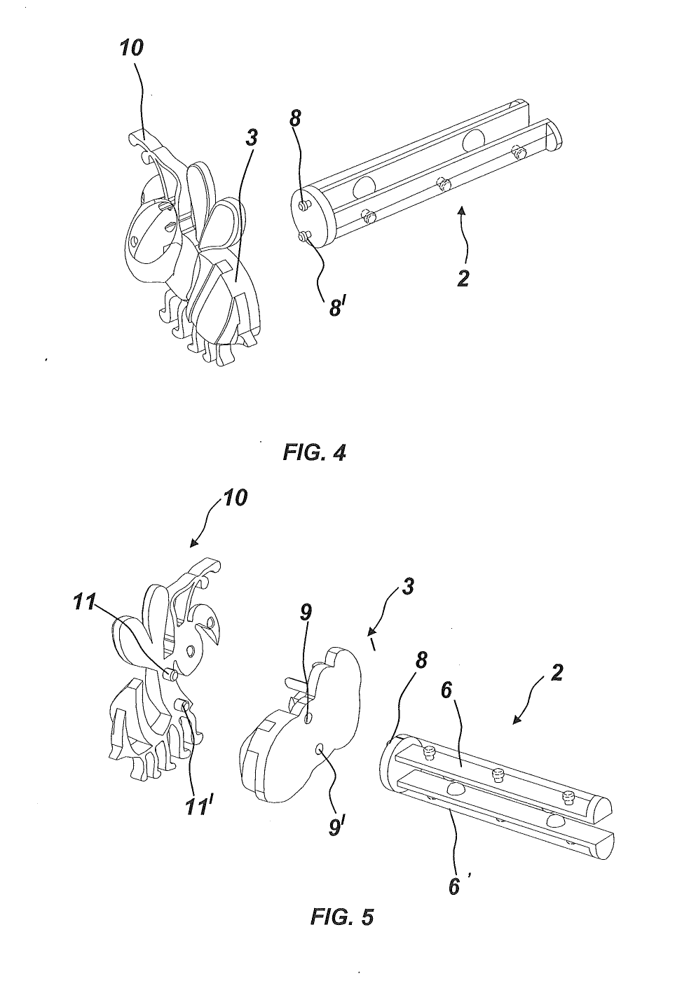

FIGS. 4 and 5 show a further embodiment of an air freshener 1, in which the first decorative air freshening element 3 partially defines the figure of a bee and the second decorative element 10 defines the complementary part of the same figure. The two elements 3 and 10 may be coupled together by means of a pair of pins 11, 11′ formed on the second decorative element 10 that can be inserted into complementary female seats formed on the first decorative scent element 3.

The above disclosure clearly shows that the invention fulfills the intended objects and particularly meets the requirement of providing an air freshener that has a longer-lasting high deodorizing power.

The air freshener of the invention is susceptible to a number of changes and variants, within the inventive concept disclosed in the appended claims. All the details thereof may be replaced by other technically equivalent parts, and the materials may vary depending on different needs, without departure from the scope of the invention.

While the air freshener has been described with particular reference to the accompanying figures, the numerals referred to in the disclosure and claims are only used for the sake of a better intelligibility of the invention and shall not be intended to limit the claimed scope in any manner.

The invention claimed is:

1. An air freshener, particularly for motor vehicles, comprising:
a support element adapted to be at least partially inserted in a concealable manner into an air grille of a space to be deodorized; and
at least one first decorative element associated with said support element so as to be visible from outside the air grille when the support element is in its operating position,
wherein said at least one decorative element is made from a first polymer material, which includes a thermoplastic polymer matrix and a scent agent dispersed therein for deodorizing an environment.

2. The air freshener as claimed in claim 1, wherein said scent agent in included in said first material from 20% to 40 based on total weight of the first polymer material.

3. The air freshener as claimed in claim 1, wherein said scent agent is selected from the group consisting of natural and synthetic scent materials, natural essences, and essential oils.

4. The air freshener as claimed in claim 1, wherein said polymer material is included in said first material from 80% to 60% based on total weight of the first material.

5. The air freshener as claimed in claim 1, wherein said polymer matrix is an EVA-based matrix.

6. The air freshener as claim 1, wherein said first material consists of said thermoplastic polymer matrix and said scent agent.

7. The air freshener as claimed in claim 1, wherein said at least one first decorative element is formed by molding of said first polymer material.

8. The air freshener as claimed in claim 1, wherein said at least one first decorative element is removably associated with said support element.

9. The air freshener as claimed in claim 8, further comprising a male-female member removably associating said at least one first decorative element with said support element.

10. The air freshener as claimed in claim 1, further comprising at least one second decorative element associated with said at least one first decorative element, so as to be at least partially visible from outside by a user.

11. The air freshener as claimed in claim 10, wherein said at least one first decorative element has a first pigmentation, said at least one second decorative element having a pigmentation different from the first pigmentation.

12. The air freshener as claimed in claim 10, wherein said first decorative element and said second decorative element are removably associated with each other.

13. The air freshener as claimed in claim 10, wherein said first decorative element and said second decorative element have mating shapes.

14. The air freshener as claimed in claim 1, wherein said support element comprises at least one substantially elongate arm.

15. The air freshener as claimed in claim 14, wherein said at least one elongate arm has means for attachment thereof to an air grille.

16. The air freshener as claimed in claim 14, wherein at least one second air freshening element is provided, in association with said at least one elongate arm.

17. The air freshener as claimed in claim 1, wherein said support element comprises a pair of substantially parallel elongate arms, each arm of said pair having a corresponding second air freshening element.

18. The air freshener as claimed in claim 17, wherein each arm has corresponding means for attachment thereof to the air grille.

19. The air freshener as claimed in claim 18, wherein said means for attachment to the air grille comprise mutually facing transverse bulges formed on an inner side of said arms.

20. The air freshener as claimed in claim 19, wherein said bulges are axially spaced, such that the bulges of one arm face the bulges of the other arm.
